(19) United States
(12) Patent Application Publication
(10) Pub. No.: US 2026/0096896 A1
(43) Pub. Date: Apr. 9, 2026

(54) MODULAR GLENOID IMPLANT

(71) Applicant: Bradley Carofino, Virginia Beach, VA (US)

(72) Inventor: Bradley Carofino, Virginia Beach, VA (US)

(21) Appl. No.: 18/907,275

(22) Filed: Oct. 4, 2024

(51) Int. Cl. A61F 2/40; A61F 2/30

(52) U.S. Cl. CPC A61F 2/4081; A61F 2002/30604

(57) ABSTRACT

A novel shoulder replacement system that utilizes multiple small implant plates instead of a single large one. The modular implant pieces are strategically placed in a shoulder joint to reduce the “rocking horse phenomenon,” preserve bone stock, and be minimally invasive. In addition, the system allows for customizable fit to accommodate a patient's specific wear pattern.

### TECHNICAL FIELD

The present disclosure relates generally relates to medical devices, specifically to implants used in shoulder replacement surgery.

### DESCRIPTION OF RELATED ART

Shoulder replacement surgery, also known as shoulder arthroplasty (TSA), is a common procedure performed to relieve pain and restore function in patients suffering from severe shoulder joint conditions such as osteoarthritis, rheumatoid arthritis, post-traumatic arthritis, rotator cuff tear arthropathy, or severe fractures. Traditional shoulder implants typically consist of a metal ball attached to a stem and a plastic socket, which replace the damaged humeral head and glenoid socket, respectively.

Despite advancements in shoulder implant technology, several issues persist that can compromise the success of the surgery and patient satisfaction. One issue includes loosening of the implant. One of the most significant challenges is the loosening of the implant components over time, which can lead to instability and pain, often necessitating revision surgery. Another issue is bone preservation. Preserving bone stock is crucial for the longevity of the implant and for potential future surgeries. Some current designs require substantial bone removal, which can complicate revision procedures. A third issue is surgical invasiveness. The existing sockets tend to be larger implants. The amount of surgical exposure necessary to implant these larger implants is substantial, thereby making minimally invasive surgery very difficult. Thus, there is a need for an improved shoulder implant design.

### SUMMARY

The following presents a simplified summary of the disclosure in order to provide a basic understanding of certain embodiments of the present disclosure. This summary is not an extensive overview of the disclosure and it does not identify key/critical elements of the present disclosure or delineate the scope of the present disclosure. Its sole purpose is to present some concepts disclosed herein in a simplified form as a prelude to the more detailed description that is presented later.

Aspects of the present disclosure relate to a shoulder implant, a system, and a method for shoulder implant surgery. The shoulder implant, the system, and the method all include a plurality of modular glenoid implant pieces. Each implant piece in the plurality of modular glenoid implant pieces is a separate and distinct piece. Each implant piece is configured to be implanted in a glenoid socket such that contact between a humeral head replacement and native glenoid bone in the glenoid socket is reduced in comparison to the humeral head replacement in a natural glenoid socket without the shoulder implant. Each implant piece in the plurality of modular implant pieces is also configured such that loosening of each implant piece over time, when surgically implanted into native glenoid bone, is reduced in comparison to a shoulder implant consisting of a singular glenoid implant piece.

In some embodiments, the plurality of modular glenoid implant pieces is surgically implanted into the glenoid socket such that the humeral head replacement will never contact native glenoid bone. In some embodiments, the plurality of modular glenoid implant pieces is surgically implanted into the glenoid socket such that native glenoid bone in the center of the glenoid socket is preserved. In some embodiments, the plurality of modular glenoid implant pieces is surgically implanted into the glenoid socket in a circular configuration. In some embodiments, each implant piece in the plurality of modular glenoid implant pieces is circular in shape. In some embodiments, the plurality of modular glenoid implant pieces is surgically implanted into the glenoid socket in a triangular configuration. In some embodiments, the plurality of modular glenoid implant pieces is surgically implanted into the glenoid socket in a square configuration.

These and other embodiments are described further below with reference to the figures.

### BRIEF DESCRIPTION OF THE DRAWINGS

The disclosure may best be understood by reference to the following description taken in conjunction with the accompanying drawings, which illustrate particular embodiments.

FIGS. 1A-1B show an example of a standard plastic socket replacement, in accordance with embodiments of the present disclosure.

FIGS. 2A-2B illustrate an example of an improved implant, in accordance with embodiments of the present disclosure.

FIGS. 3A-3B illustrate vertical slice views of examples of a shoulder socket with different implants, in accordance with embodiments of the present disclosure.

FIGS. 4A-4C illustrate three different types of implants, in accordance with embodiments of the present disclosure.

FIGS. 5A-5C illustrate an example of a surgery technique utilizing the implant, configured in accordance with embodiments of the present disclosure.

FIG. 6 illustrates a method for shoulder implant surgery, in accordance with embodiments of the present disclosure.

### DESCRIPTION OF EXAMPLE EMBODIMENTS

Reference will now be made in detail to some specific examples of the present disclosure including the best modes contemplated by the inventors for carrying out the present disclosure. Examples of these specific embodiments are illustrated in the accompanying drawings. While the present disclosure is described in conjunction with these specific embodiments, it will be understood that it is not intended to limit the present disclosure to the described embodiments. On the contrary, it is intended to cover alternatives, modifications, and equivalents as may be included within the spirit and scope of the present disclosure as defined by the appended claims.

For example, portions of the techniques of the present disclosure will be described in the context of shoulder implant surgery. However, it should be noted that the techniques of the present disclosure may apply to a wide variety of different types of implants, including hips and knees. In the following description, numerous specific details are set forth in order to provide a thorough understanding of the present disclosure. Particular example embodiments of the present disclosure may be implemented without some or all of these specific details. In other instances, well known process operations have not been described in detail in order not to unnecessarily obscure the present disclosure.

Various techniques and mechanisms of the present disclosure will sometimes be described in singular form for clarity. However, it should be noted that some embodiments include multiple iterations of a technique or multiple instantiations of a mechanism unless noted otherwise. For example, an implant can use a plug in a variety of contexts. However, it will be appreciated that an implant can use multiple plugs while remaining within the scope of the present disclosure unless otherwise noted. Furthermore, the techniques and mechanisms of the present disclosure will sometimes describe a connection between two entities. It should be noted that a connection between two entities does not necessarily mean a direct, unimpeded connection, as a variety of other entities may reside between the two entities. Consequently, a connection does not necessarily mean a direct, unimpeded connection unless otherwise noted.

As mentioned above, the shoulder joint, comprising the ball (humeral head) and socket (glenoid), is a complex and vital structure prone to degeneration, especially from arthritis. Arthritis leads to the erosion of the cartilage covering the humeral head and glenoid, resulting in painful bone-on-bone contact. As this wear progresses, the glenoid often becomes misshapen, exacerbating joint dysfunction and discomfort.

Total shoulder arthroplasty (TSA), or shoulder replacement surgery, is a common and effective treatment for severe shoulder arthritis. TSA involves replacing the damaged humeral head with a metal ball prosthesis and covering the glenoid with a plastic socket. While TSA typically results in excellent clinical outcomes, several significant issues persist with the current plastic socket designs.

One major problem is the potential for implant failure over time, which often necessitates revision surgeries. The primary mode of failure is caused by micromotion of the socket at the anchoring points, leading to glenoid bone loss and loosening of the implant. This mechanism, known as the “rocking horse” phenomenon, occurs when pressure on one side of the socket causes the opposite side to lift off, much like a rocking horse. This repetitive motion undermines the stability of the implant, leading to its eventual failure.

Another critical issue with current socket designs is the significant removal of bone from the central glenoid. This area is crucial for the success of any potential future revision surgeries. Excessive bone loss in this region can complicate or even render impossible the revision procedures, limiting long-term patient outcomes.

Furthermore, the size and design of current socket implants make minimally invasive surgery very challenging. These larger implants require substantial surgical exposure for proper placement, which can involve extensive incisions and muscle cutting. This level of invasiveness increases recovery time and the risk of complications, making the procedure more taxing on patients.

The techniques and mechanisms of the present disclosure provide for an improved glenoid implant design that has been developed to address these prevalent issues. The improved design employs multiple smaller implant pieces rather than a single large component. This innovative design reduces the forces that cause the rocking horse effect, thereby minimizing the risk of implant loosening. Additionally, the improved design's modular nature allows for bone preservation in the critical central glenoid area, facilitating easier and more successful revision surgeries if needed. Moreover, the smaller size of the improved implant pieces allows for minimally invasive surgical techniques, reducing the required surgical exposure and potentially improving recovery times.

The design of the techniques and mechanisms of the present disclosure represents a significant departure from traditional glenoid implant designs. Conventional designs typically involve a single plastic piece that covers most of the glenoid bone surface. The improved design, however, consists of several smaller, individual plastic implants that can be strategically placed to cover specific portions of the glenoid bone surface. This modular approach offers several key advantages.

Firstly, the use of smaller, unconnected implants significantly reduces or eliminates the rocking horse phenomenon, thereby decreasing the likelihood of glenoid failure. By minimizing the distance between points on the implant, the design reduces the lever arm forces that contribute to implant loosening. This enhancement improves the long-term stability and durability of the implant.

Secondly, the improved design preserves the central glenoid bone. This preservation is crucial for potential future revision surgeries, as maintaining bone stock in this critical area is essential for securing new implants. By arranging the implant components to avoid excessive central bone removal, the design ensures better outcomes for patients who may require additional surgeries.

Thirdly, the smaller size of the implants facilitates minimally invasive surgery. Implanting smaller pieces requires less surgical exposure than traditional larger implants. This reduction in required exposure allows for smaller incisions and less muscle cutting, which can significantly improve patient recovery times and reduce the overall risk of surgical complications.

Historically, various glenoid design iterations have been explored to enhance implant fixation and reduce micromotion and failure. These designs can be broadly categorized into three main types: standard glenoids, inset glenoids, and inlay glenoids.

Standard glenoids involve a plastic socket that sits atop the glenoid and is secured by pegs or other fixation points. Modifications to this design have included altering the number, size, and shape of these fixation points and incorporating surface modifications to promote bone ingrowth.

Inset glenoids, on the other hand, are partially recessed within the bone. This design offers biomechanical advantages that can decrease loosening but often requires substantial bone removal from the central glenoid.

Inlay glenoids sit entirely within the bone and, like inset designs, necessitate significant central bone removal. While these designs offer certain biomechanical benefits, the extensive bone removal can complicate revision procedures.

As previously mentioned, the techniques and mechanisms of the present disclosure introduce a novel approach by using multiple smaller pieces, which can be applied to all three design styles: standard, inset, and inlay. When integrated into each of these styles, the improved implant offers distinct advantages, such as enhanced fixation, reduced bone removal, and the potential for minimally invasive surgery, making it a superior option for shoulder arthroplasty.

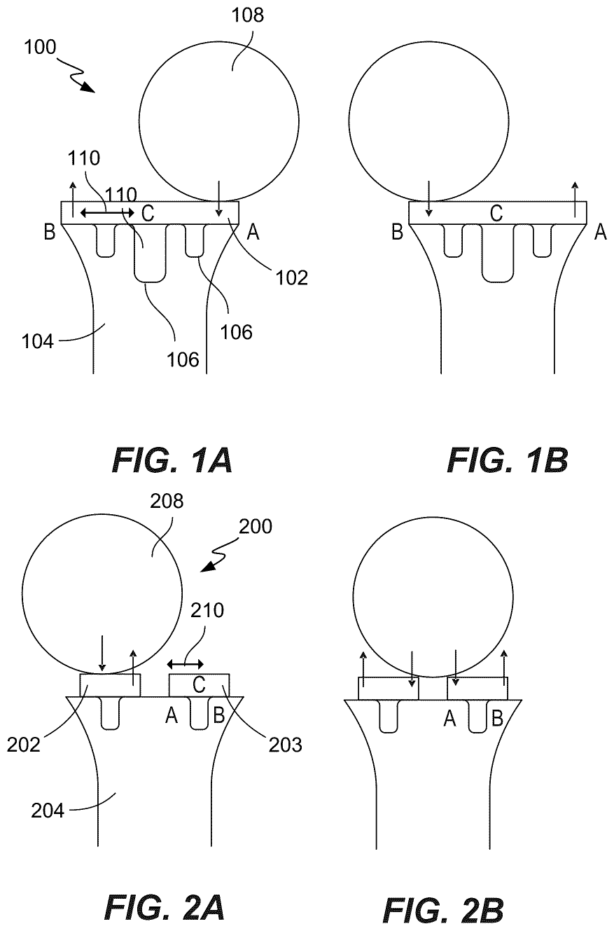

FIGS. 1A-1B show an example of a standard plastic socket replacement, in accordance with embodiments of the present disclosure. Replacement joint 100 shows a plastic socket replacement 102 sitting on top of a bone socket 104. In some embodiments, plastic socket replacement 102 includes pegs 106 that anchor into the bone. In some embodiments, replacement joint 100 also includes a humeral head 108. Humeral head 108 is typically a ball-shaped replacement of a natural humeral head.

FIGS. 1A-1B illustrate an example of the “rocking horse” phenomenon. As shown in FIGS. 1A-1B, humeral head 108 sits on plastic socket replacement 102 and the “ball” slides back and forth from one edge of the socket (“A” as shown in FIG. 1A) to the other (“B” as shown in FIG. 1B). When the ball gets to one edge of the socket, there is a “seesaw” phenomenon. In other words, as humeral head 108 applies a downward force onto plastic socket replacement 102 at edge A, the opposite side (edge B) lifts up, as shown in FIG. 1A. Similarly, when humeral head 108 reaches the other side of plastic socket replacement 102, it applies a downward force on edge B, thereby causing edge A to lift up, as shown in FIG. 1B. Because humeral head 108 slides back and forth, as previously described, thousands of times per day, it is continually causing those forces of lift off on either other side of plastic socket replacement 102. Over time, this back and forth “rocking” motion leads to the socket loosening and failing. Although in most replacements, there are pegs 106 on the back of plastic socket replacement 102 that secure attachment into the bone, most replacements will generally fail over a long enough period of time. This phenomenon is unique to plastic socket replacements sitting on top of the bone. When humeral head 108 has high downward contact pressure on one edge of plastic socket replacement 102, a lift up force is applied to the other edge. The amount of force is defined by the lever arm 110. The lever arm is defined from the center of the plastic socket replacement 102 (“C”) to either edge. Because the lever arm is defined by distance from the center to the edge, the smaller the distance, the smaller the lever arm. The smaller the lever arm, the less lifting force is applied to the edges. This would ultimately result in a reduction of the rocking horse phenomenon, or potentially the elimination of the phenomenon entirely.

FIGS. 2A-2B illustrate an example of an improved implant, in accordance with embodiments of the present disclosure. As shown in FIG. 2A, improved replacement joint 200 includes several strategically placed modular plastic socket replacement pieces 202 and 203. In some embodiments, when humeral head 208 reaches one edge of bone 204, it sits entirely on modular replacement piece 202. When this occurs, the downward force is applied at the point of contact on modular replacement piece 202. However, because the replacement piece itself is smaller, then the lifting force is also smaller because of a shorter lever arm 210. In addition, there is no force at all on modular replacement piece 203.

Conventional wisdom for a joint replacement is to try to cover the entire joint. The reason for this is to protect the bone from wear and tear resulting from contact with an artificial humeral head. Thus, it is believed to be “safer” just to use a single large replacement piece. However, this leads to the problems mentioned above. The techniques and mechanisms of the present disclosure include many modular small replacement pieces to prevent the rocking horse phenomenon. However, the pieces must be strategically place such that parts of the bone are not at risk of contact exposure with the humeral head. If modular replacement pieces are strategically arranged, the multiple small pieces still allow for an air gap between adjacent pieces, but the humeral head never touches the joint surface even if the entire surface of the bone is not completely covered.

FIG. 2B shows humeral head 208 sitting directly on top of the air gap between modular replacement pieces 202 and 203. Although humeral head 208 sits on top of the air gap, it does not actually touch the surface of bone 204. In addition, although there is a downward force exerted on the edge of each module replacement piece, the lifting force on the opposite side of each modular replacement piece is less because the lever arms of each piece is smaller and the force is distributed among two different pieces. In other words, using strategically placed modular replacement pieces 202 and 203 reduces the lifting forces, as compared to a single replacement piece, that would cause the rocking horse phenomenon on each piece, while still functioning the same as a single replacement piece 102.

In addition to reducing the forces that cause the rocking horse phenomenon, using multiple modular implant pieces allows for more preservation of bone, especially the bone in the center of the socket, which may be important for potential revision surgeries. When replacements fail, it is necessary to have good bone remaining in the central portion of socket to convert to a new type of replacement. If a first replacement is placed in the center of the bone, and the bone was drilled away, then when a second replacement needs to be performed, there is less bone in the center. Thus, in a replacement design using multiple modular replacement pieces, the pieces can be strategically placed in a pattern that preserves the center bone. In such embodiments, the humeral head floats on top of the pieces peripherally, but yet never touches the central bone.

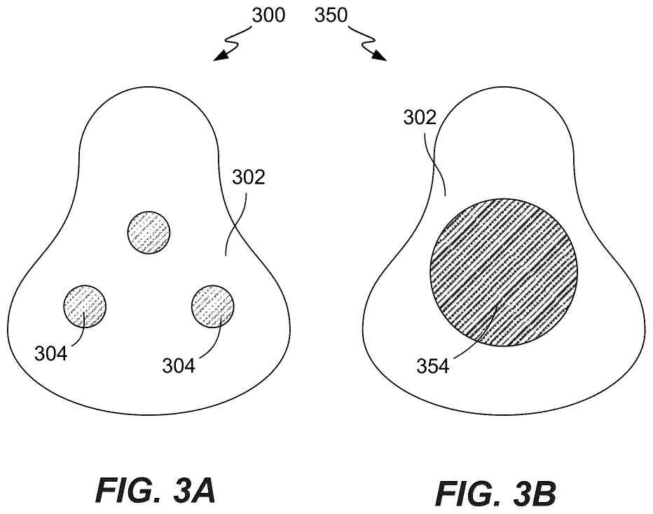

FIGS. 3A-3B illustrate vertical slice views of examples of a shoulder socket with different implants, in accordance with embodiments of the present disclosure. As shown in FIGS. 3A and 3B, the socket of the shoulder naturally is shaped like a pear. FIGS. 3A and 3B show a vertical slice view of socket 300 and 350, as if the humeral head was removed entirely and the viewer was looking direction into the shoulder sockets.

In both figures, the white area is bone 302. However, the shaded areas represents different types of implants. FIG. 3B shows socket 350, which has been implanted with a type of replacement called an inlay glenoid 354. Previous FIGS. 1A-2B showed socket implants on top of the bone. An inlay glenoid is a different type of socket implant where the socket implant sits inside the bone, e.g., flush like a manhole cover on a road.

The same concept can be applied and improved with the techniques and mechanisms of the present disclosure. FIG. 3A shows socket 300 with modular glenoid implants 304 that essentially work as an inlay glenoid. In such an embodiment, modular implant pieces 304 can be arranged around the central bone. In some embodiments, modular implant pieces 304 can be mostly inlaid (e.g., over 50-95% inlaid) with only a tiny bit of the modular implant pieces sticking out of the bone right about the surface of bone 302. In such embodiments, the humeral head would not touch the bone at all, assuming the modular implant pieces 304 were arranged just wide enough to allow for maximum bone preservation while still preventing the humeral head from touching the central bone. Since such embodiments are only partially inlaid, the design of such embodiments is more of a hybrid between top-setting and inlaid implants.

In some embodiments, modular implant pieces 304 can also be completely flush. However, such a design does not prevent the humeral head from rubbing against central bone. Although some people's preference would be to completely inlay the modular implant pieces for stability purposes, the hybrid approach appears to be more advantageous. In embodiments where the modular implant pieces were completely inlaid, there would still be advantages over the standard inlaid glenoid as shown in FIG. 3B. For example, having modular implant pieces 304 arranged around the central bone would still preserve the central bone for future revision surgeries. Although the inlaid glenoid illustrated in FIG. 3B does protect rubbing against the central bone, the design still allows for rubbing against the periphery bone and yet the central bone is not preserved. Without preserving the central bone, a revision replacement surgery requires a reverse replacement and insertion of a big screw right down the middle of the socket. With the techniques and mechanisms of the present disclosure, there will still be bone available in the middle for a revision replacement.

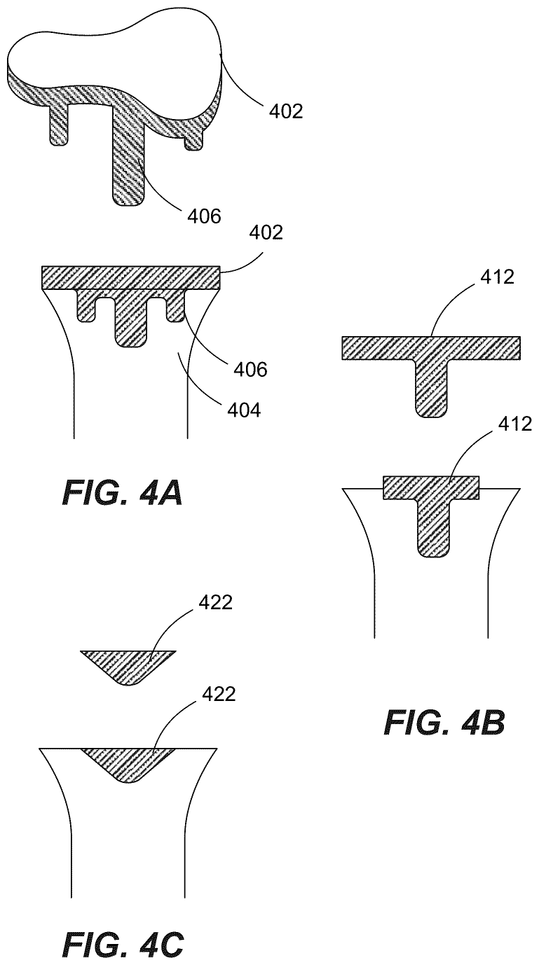

As shown in the above examples, the modular implant design is extremely versatile and can be used in a variety of ways. FIGS. 4A-4C illustrate three different types of modular implants, in accordance with embodiments of the present disclosure. For illustration purposes, FIGS. 4A-4C show the type of implant only, and not the modular design described throughout the present disclosure. It should be understood that the modular glenoid implant design can also be applied using any of the three different types of implants described in FIGS. 4A-4C.

FIG. 4A illustrates the standard top setting type of implant. As shown in FIG. 4A, top setting implant 402 is configured such that the plastic fits entirely on top of bone 404. In some embodiments, top setting implant 402 is anchored in place by pegs 406 that go directly into the bone 404.

FIG. 4B illustrates the hybrid type implant design. As shown in FIG. 4B, inset glenoid 412 is partially inlaid in the bone. In some embodiments, inset implant 412 is essentially recessed into the bone. Such embodiments can provide a biomechanical advantage to being partly inside the bone. More specifically, inset implant 412 is less likely to loosen, but still protects the bone from rubbing contact with the humeral head.

FIG. 4C illustrates the inlay type implant design. As shown in FIG. 4C, inlay glenoid 422 sits entirely inside the bone. In some embodiments, inlay implant 422 is completely flush with the outside surface of the bone. In such embodiments, inlay implant 422 encounters less rocking horse phenomenon because when the humeral head reaches the far edge of inlay implant 422, inlay implant 422 is supported by the bone. However, the disadvantage of using inlay implant 422 is the rubbing of the humeral head on bone along the edges, as well as the non-trivial removal of central bone.

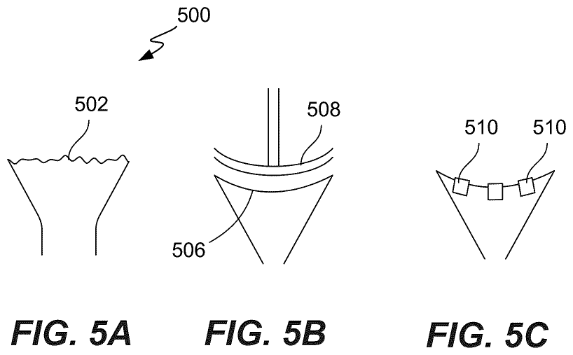

As mentioned above with reference to FIGS. 4A-4C, the techniques and mechanisms of the present disclosure can be used in a variety of ways. FIGS. 5A-5C illustrate an example of a surgery technique utilizing the implant, configured in accordance with embodiments of the present disclosure. FIG. 5A illustrates what can happen to socket 500 that has been affected by arthritis. As shown in FIG. 5A, socket 500 eventually becomes deformed. In many instances of arthritis, the surface 502 has a rough area where the humeral head (not shown) would be sitting. One type of technique for addressing this condition is called a “ream and run.” The idea of a ream and run is to reshape the rough surface area 504 and make it a smooth surface 506, as shown in FIG. 5B. Typically, this is performed using a tool called a reamer 508. Reamer 508 is designed to smooth out rough surface areas of the bone.

Finally, once surface 506 is smooth, the surface is implanted with multiple small modular pieces 510, which are used to replace multiple small areas of the bone. In some embodiments, these modular pieces 510 can be inset type or inlaid type. The advantage is that now when the metal humeral head is placed in the socket, not all the force is going to the bone. Instead, the modular pieces act as multiple areas of shock absorbers. Without these shock absorbers, having metal rub on bone could cause pain and erosion over time. Thus, putting in the modular plastic pieces could alleviate these future problems. It should be appreciated that although plastic, e.g., polyethylene, is the most common material used in replacements, the techniques and mechanisms of the present disclosure could be applied to other various material forms that are currently used for shoulder replacement, such as ceramics, metal, and any other material suitable for implant use.

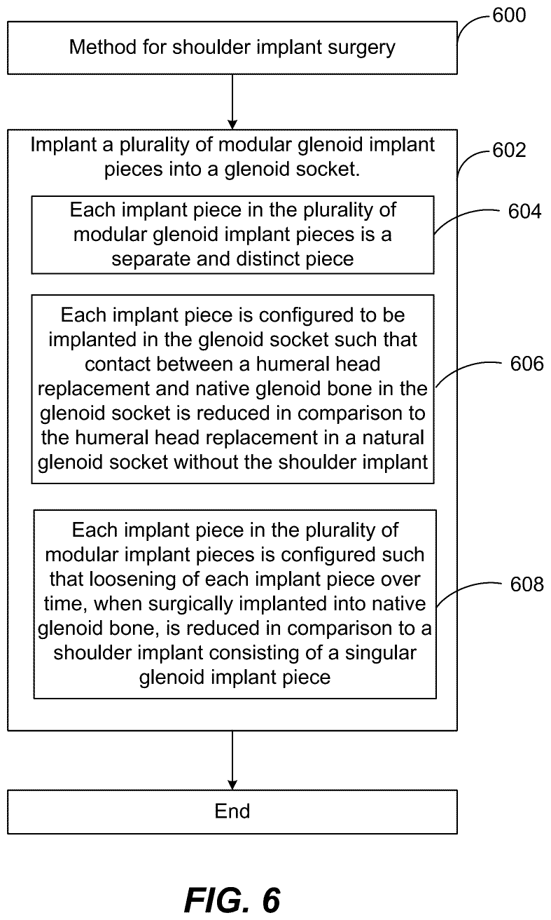

FIG. 6 illustrates a method for shoulder implant surgery, in accordance with embodiments of the present disclosure. a method for shoulder implant surgery. Method 600 includes step 602. At step 602, a plurality of modular glenoid implant pieces is implanted into a glenoid (shoulder) socket. In some embodiments, each implant piece in the plurality of modular glenoid implant pieces is a separate and distinct piece (604). In some embodiments, each implant piece is configured to be implanted in a glenoid socket such that contact between a humeral head replacement and native glenoid bone in the glenoid socket is reduced in comparison to the humeral head replacement in a natural glenoid socket without the shoulder implant (606). In some embodiments, each implant piece in the plurality of modular implant pieces is also configured such that loosening of each implant piece over time, when surgically implanted into native glenoid bone, is reduced in comparison to a shoulder implant consisting of a singular glenoid implant piece (608).

In some embodiments, the plurality of modular glenoid implant pieces is surgically implanted into the glenoid socket such that the humeral head replacement will never contact native glenoid bone. In some embodiments, the plurality of modular glenoid implant pieces is surgically implanted into the glenoid socket such that native glenoid bone in the center of the glenoid socket is preserved. In some embodiments, the plurality of modular glenoid implant pieces is surgically implanted into the glenoid socket in a circular configuration. In some embodiments, each implant piece in the plurality of modular glenoid implant pieces is circular in shape. In some embodiments, the plurality of modular glenoid implant pieces is surgically implanted into the glenoid socket in a triangular configuration. In some embodiments, the plurality of modular glenoid implant pieces is surgically implanted into the glenoid socket in a square configuration.

According to various embodiments, one important reason for substituting a large singular implant, which is the industry standard, with a plurality of implant pieces is to distribute load sharing among the different pieces. In addition, the implant pieces act as a barrier between the humeral head replacement and native humeral bone, thereby reducing force and friction exerted onto the bone in order to reduce erosion, which leads to pain because it's wearing into the bone.

The techniques and mechanisms of the present disclosure provide many benefits over conventional shoulder replacement technology, e.g., reduction/elimination of the rocking horse phenomenon, preservation of the central bone, and reducing invasiveness of surgery, because the smaller pieces used in the implant translates to smaller cuts needed for surgery. Yet another advantage of using modular glenoid implants as described herein is the ability to customize to the implant to a particular situation. Because every individual is different, people can have different wear patterns of the socket. This means that different areas of a shoulder become worn depending on the individual. In other words, patient A that needs a shoulder replacement surgery may have different areas of the socket that need to be replaced than patient B who also needs shoulder replacement surgery. Using the techniques and mechanisms of the present disclosure, it is possible to simply replace those portions that are worn, instead of the entire joint.

In addition, in conventional replacement surgery, implants are specifically designed to replace a particular area of a joint. In other words, the implant is designed to replace a portion of the joint. However, the techniques and mechanisms of the present disclosure utilize a different, more effective approach. Instead of trying to replace a portion of the joint, the idea behind modular implant design is to provide an ideal shape for coupling with an artificial humeral head. In other words, the modular implant embodiments disclosed herein are specifically designed to optimally couple with a replacement humeral head, rather than attempt to restore the joint surface. Instead of trying to recreate the shape the joint, a new structure is implanted specifically designed and customized to fit with the replacement humeral head. In many embodiments, the shape of this new plastic socket is not the same shape as a natural socket. This is because in a natural shoulder joint, the cup of the shoulder is formed by a combination of bone, ligaments, labrum, etc. Conventional shoulder replacement surgery uses a one-size-fits-all design, which does not take into account inherent defects/deviations in the shape/curvature of the socket. By contrast, the techniques and mechanisms of the present disclosure provide for modular implants perfectly suited for customization to account for these issues.

In the foregoing specification, the present disclosure has been described with reference to specific embodiments. However, one of ordinary skill in the art appreciates that various modifications and changes can be made without departing from the scope of the present disclosure as set forth in the claims below. Accordingly, the specification and figures are to be regarded in an illustrative rather than a restrictive sense, and all such modifications are intended to be included within the scope of present disclosure.

What is claimed is:

1. A shoulder implant comprising:
a plurality of modular glenoid implant pieces, wherein each implant piece in the plurality of modular glenoid implant pieces is a separate and distinct piece,
wherein each implant piece is configured to be implanted in a glenoid socket such that contact between a humeral head replacement and native glenoid bone in the glenoid socket is reduced in comparison to the humeral head replacement in a natural glenoid socket without the shoulder implant,
wherein each implant piece in the plurality of modular implant pieces is configured such that loosening of each implant piece over time, when surgically implanted into native glenoid bone, is reduced in comparison to a shoulder implant consisting of a singular glenoid implant piece.

2. The shoulder implant of claim 1, wherein the plurality of modular glenoid implant pieces is surgically implanted into the glenoid socket such that the humeral head replacement will never contact native glenoid bone.

3. The shoulder implant of claim 2, wherein the plurality of modular glenoid implant pieces is surgically implanted into the glenoid socket such that native glenoid bone in the center of the glenoid socket is preserved.

4. The shoulder implant of claim 1, wherein the plurality of modular glenoid implant pieces is surgically implanted into the glenoid socket in a circular configuration.

5. The shoulder implant of claim 1, wherein each implant piece in the plurality of modular glenoid implant pieces is circular in shape.

6. The shoulder implant of claim 1, wherein the plurality of modular glenoid implant pieces is surgically implanted into the glenoid socket in a triangular configuration.

7. The shoulder implant of claim 1, wherein the plurality of modular glenoid implant pieces is surgically implanted into the glenoid socket in a square configuration.

8. A system comprising:
a plurality of modular glenoid implant pieces, wherein each implant piece in the plurality of modular glenoid implant pieces is a separate and distinct piece,
wherein each implant piece is configured to be implanted in a glenoid socket such that contact between a humeral head replacement and native glenoid bone in the glenoid socket is reduced in comparison to the humeral head replacement in a natural glenoid socket without the shoulder implant,
wherein each implant piece in the plurality of modular implant pieces is configured such that loosening of each implant piece over time, when surgically implanted into native glenoid bone, is reduced in comparison to a shoulder implant consisting of a singular glenoid implant piece.

9. The system of claim 8, wherein the plurality of modular glenoid implant pieces is surgically implanted into the glenoid socket such that the humeral head replacement will never contact native glenoid bone.

10. The system of claim 9, wherein the plurality of modular glenoid implant pieces is surgically implanted into the glenoid socket such that native glenoid bone in the center of the glenoid socket is preserved.

11. The system of claim 8, wherein the plurality of modular glenoid implant pieces is surgically implanted into the glenoid socket in a circular configuration.

12. The system of claim 8, wherein each implant piece in the plurality of modular glenoid implant pieces is circular in shape.

13. The system of claim 8, wherein the plurality of modular glenoid implant pieces is surgically implanted into the glenoid socket in a triangular configuration.

14. The system of claim 8, wherein the plurality of modular glenoid implant pieces is surgically implanted into the glenoid socket in a square configuration.

15. A method for shoulder implant surgery, the method comprising:
implanting a plurality of modular glenoid implant pieces into a glenoid socket, wherein each implant piece in the plurality of modular glenoid implant pieces is a separate and distinct piece,
wherein each implant piece is configured to be implanted in the glenoid socket such that contact between a humeral head replacement and native glenoid bone in the glenoid socket is reduced in comparison to the humeral head replacement in a natural glenoid socket without the shoulder implant,
wherein each implant piece in the plurality of modular implant pieces is configured such that loosening of each implant piece over time, when surgically implanted into native glenoid bone, is reduced in comparison to a shoulder implant consisting of a singular glenoid implant piece.

16. The method of claim 15, wherein the plurality of modular glenoid implant pieces is surgically implanted into the glenoid socket such that the humeral head replacement will never contact native glenoid bone.

17. The method of claim 16, wherein the plurality of modular glenoid implant pieces is surgically implanted into the glenoid socket such that native glenoid bone in the center of the glenoid socket is preserved.

18. The method of claim 15, wherein the plurality of modular glenoid implant pieces is surgically implanted into the glenoid socket in a circular configuration.

19. The method of claim 15, wherein each implant piece in the plurality of modular glenoid implant pieces is circular in shape.

20. The method of claim 15, wherein the plurality of modular glenoid implant pieces is surgically implanted into the glenoid socket in a triangular configuration.
